(19) United States
(12) Patent Application Publication
(10) Pub. No.: US 2007/0042967 A1
(43) Pub. Date: Feb. 22, 2007

(54) Proteaseome inhibitors for the treatment of herpesviridae infected individuals

(75) Inventors: Susanne Proesch, Berlin (DE); Hans Dieter Volk, Berlin (DE); Detlev Krueger, Berlin (DE)

Correspondence Address: ROTHWELL, FIGG, ERNST & MANBECK, P.C., 1425 K STREET, N.W., SUITE 800, WASHINGTON, DC, 20005, US

(73) Assignee: Charite-Universitaetsmedizin Berlin, Berlin (DE)

(21) Appl. No.: 11/528,361

(22) Filed: Sep. 28, 2006

Related U.S. Application Data
(63) Continuation of application No. 10/520,150, filed on Mar. 22, 2005, now abandoned.

(30) Foreign Application Priority Data
Jul. 3, 2002 (EP) 02 014 728.6

(51) Int. Cl. A61K 38/05; A61K 38/04; A61K 31/69

(52) U.S. Cl. 514/18; 514/19; 514/64

(57) ABSTRACT

The present invention relates to the use of a substance or composition comprising one or more proteasome inhibitors for the manufacture of a medicament for the treatment of an individual infected with a virus selected from the group comprising varicella zoster virus, human cytomegalovirus, human herpesvirus 6 and 7 and Epstein-Barr virus and Karposi's sarcoma herpesvirus. The invention further relates to methods of treatment of individuals infected with a virus selected from the group comprising varicella zoster virus, human cytomegalovirus, human herpesvirus 6 and 7 and Epstein-Barr virus and Karposi's sarcoma herpesvirus.

This application is a continuation of U.S. Ser. No. 10/520,150 filed Mar. 22, 2005, which is a 35 USC § 371 National Phase Entry Application from PCT/EP03/07062, filed Jul. 2, 2003, and designating the U.S.

### BACKGROUND OF THE INVENTION

Herpesviridae is the name of a family of enveloped, double-strained DNA viruses with relatively large genomes. They replicate in the nucleus of a wide range of invertebrate hosts, including eight varieties isolated in humans, several each in horses, cattle, mice, pigs, chickens, turtles, lizards, fish and even in some invertebrates such as oysters. Human herpesviridae infections are endemic and sexual contact is a significant method of transmission for several including both herpes simplex virus 1 and 2(HSV-1, HSV-2, HHV1 and HHV2), also human cytomegalovirus (HCMV, HHV5) and likely Karposi's sarcoma herpesvirus (HHV-8). Four biological properties characterize members of the herpesviridae family:

Herpesviruses express a large number of enzymes involved in metabolism of nucleic acid (e.g. thymidinkinase), DNA synthesis (e.g. DNA helicase/primase) and processing of proteins (e.g. proteinkinase). The synthesis of viral genomes and the assembly of capsids occurs in the nucleus. Productive viral infection is accompanied by inevitable cell destruction. Herpesviruses are able to establish and maintain a latent state in their host and reactivate following cellular stress. Latency involves stable maintenance of the viral genome in the nucleus in the absence of any viral proteins (HSV1, HSV2, HCMV) or with limited expression of a small set of viral genes (VZV). In case of EBV latency the target cells become immortalized and transformed by expression of latency associated proteins.

The herpesviridae are divided into three sub-families (1) alpha-herpes-virinae, which includes herpes simplex virus 1(HHV1), herpes simplex virus 2(HHV2) and, varicella zoster virus (HHV3). (2) beta-herpes-virinae, which includes the human cytomegalovirus (HCMV, HHV5) and the human herpesviruses 6 and 7 (HHV6 and HHV7). (3) gamma-herpes-virinae which includes the Epstein-Barr virus (HHV4) and the Karposi's sarcoma herpesvirus (HHV8).

Herpes simplex virus 1 is responsible for facial, labial and ocular lesion. Herpes simplex virus 2 mainly for genital lesions. Varicella zoster virus is responsible for chickenpox shingles and zooster. The human cytomegalovirus (HCMV) can cause a wide variety of different diseases as outlined in detail further below. Epstein-Barr virus is responsible for infectious mononucleosis, lympholiferative disease as well as a cofactor in human cancers (lymphomas, carcinomas). HHV8 is a cofactor in Karposi's sarcoma development which was extremely rare until the advent of AIDS.

### Disease States in Clinical Features Associated with Human Cytomegalovirus Infection

In general, HCMV is a herpesvirus with low pathogenicity. The outcome of HCMV infection is frequently determined by the status incompetency of host immunity and, in the case of intra uterine fetal HCMV infection, the developmental stage at the time of infection. Congenital HCMV infection occurs in approximately 1% to 2% of infants in utero and another 6% to 60% of individuals become infected perinatally or postnatally during the first 6 months of life as the result of birth canal or breast milk transmission. The clinical features of symptomatic prenatal infections include hepatosplenomegaly, microencephaly, central nervous system disease, HCMV pneumonia, mental retardation and other symptoms.

Perinatal HCMV transmission often results from infectious uterine cervix, birth canal, milk and colostrum and other maternal reservoirs. Substantial proportions of infants (8% to 60%) become infected during the first months of life. On the other hand, perinatal infection can be symptomatic or even end in death if HCMV is transmitted to the immature neonate. Infections may result in symptomatic presentations such as pneumonitis, neuromuscular disability, bronchopulmonary dysplasia, and delay in speech.

HCMV infection in immuno-competent individuals is usually asymptomatic, or at the most produces a self-limited mononucleosis-like syndrome. With distinctions from Epstein-Barr virus (EBV) induced mononucleosis, HCMV mononucleosis is serologically identified to be heterophile-negative and occurs in an older age group; it is uncommon in children. The outcome of HCMV in immuno-competent patients is heavily dependent on HCMV specific protective immunities, both at the humoral and cellular levels. In congenital infection, pre-existing maternal immunity may prevent severe HCMV-induced disease.

The same is observed in organ transplant patients where the pre-existing immunity to HCMV limits diseases and syndromes associated with HCMV infection. In natural HCMV infections, immuno-competent humans respond to virus encoded envelop proteins such as capside proteins, tegument proteins and non-structural proteins. Among them, only antibodies against viral envelope glycoproteins have functional neutralization activity.

A number of structural and non-structural HCMV proteins, particularly the major tegument protein pp65 and the immediate early protein 1 (p72), have been demonstrated to provoke helper T (Tₕ) and cytotoxic/suppressor T cell (T_{c}) responses. It has been shown that MHC class I-restricted cytotoxic T-lymphocytes displaying CD8 play an important role in host defense to HCMV infection. Cytotoxic T_{c}-cells recognizing the major IE proteins of HCMV are important for recovery from acute HCMV infection and for preventing reactivation of latent virus (Borysiwicz L. K. et al. (1988) Eur. J. Immunol. 18:269-275 and Eng-Shang Huang et al., The pathogenicity of human cytomegalo virus: an overview). Also, Lindsay et al. demonstrated MHC class II (DR)-restricted cytotoxicity against HCMV in the T-lymphocyte subset with the CD4 marker. (helper/inducer) cells. Therefore, Tₕ- and T_{c}-lymphocytes are involved in MHC-cytotoxicity.

HCMV mononucleosis-like disease include malaise, headache, myalgia, protracted fever, abnormalities in liver functions, hepatosplenomegaly, and are typical lymphocytoses. In severe cases HCMV infections lead to the development of interstitial pneumonitis, subclinical myocarditis, pericarditis, acute and chronic encephalitis, aseptic meningitis, thrombocytopenic purpura, hemolytic anemia, gastroenteritis (colitis), hepatitis, retinitis and epidermolysis occur.

Furthermore, HCMV has the ability to infect different blood cell types mainly monocytes/macrophages. Infection of these cells usually results in persistent infections and altered expression of genes encoding cytokines and chemokines resulting in transient immuno-suppression

Other clinical manifestations of HCMV include gastrointestinal disease which is the most prominent manifestation of HCMV infection in a population of heart and heart-lung patients with an incidence of 9.9%, occurring most frequently in HCMV sero-negative recipients of organs of HCMV-sero-positive donors. Clinical manifestations include gastritis, duodenitis, esophagitis, pyloric perforation, colonic hemorrhage and more.

### Clinical Manifestations in Immuno-Compromised Individuals

Populations at greatest risk of HCMV infection and HCMV-induced diseases are those undergoing organ transplantation and those with malignancies receiving immuno-suppressing chemotherapy and particularly patients with AIDS. Additionally, HCMV has been shown to cause severe complications in patients with septic disease. In immuno-compromised hosts most severe and profound syndromes are observed when infected with HCMV, both as primary or recurring infections. Similarly, mortality and morbidity are also increased with HCMV infections in patients of this group. The most common sides of pathological involvement include adrenals (75%), lung (78%), gastrointestinal tract (30%), CNS (20%) and oculus (10%).

The severity, frequency and clinical manifestation of HCMV infections in transplant patients, cancer patients and other immuno-suppressed groups are quite variable. In most of the cases, mononucleosis fever is the common syndrome observed. After mononucleosis syndrome, pneumonia is the most frequent manifestation of HCMV infection in immuno-suppressed patients. It is more prevalent and severe in bone-marrow transplant patients, with mortality rates close to 40%. Chorioretinitis is the manifestation most frequently described in association with HCMV in patients with AIDS.

Because of the ubiquitous and mysterious nature of HCMV, most of the medical problems associated with HCMV have not been adequately studied. We know that severe syndromes can result from either primary, recurring infection or superinfection with another virus strain in immuno-compromised individuals or in developing fetuses. One major public health concern is that HCMV exists commonly in human semen (and in sperm) and the cervix. It can, therefore, infect fetuses, interfere with embryonic development, and cause developmental abnormalities. Induction of latency and subsequent reactivation of HCMV is comparable to that of other oncogenic herpesviruses. One may say that HCMV infection observed today in organ transplant recipients and immuno-compromised patients are much like the visible portions of icebergs.

### Infections of Particular Importance in Transplant Recipients

The most important pathogen affecting transplant recipients is HCMV, which causes both direct effects such as asymptomatic viral shedding, acute viral syndromes which are flue-like or mononucleosis-like illnesses (fever and myalgia), leucopenia, pneumonitis, infection of native tissues (retina), gastrointestinal tract (pancreas) and many more as well as indirect effects such as acute or chronic allograft rejection, immuno-suppression and more. As herpesvirus, HCMV has two properties that determine its role in transplantation: latency and cell association. Once infected (the laboratory marker of infection is sero-positivity), the patient harbors the virus for life. Activation from latency in both the recipient and the donor organ/blood is induced by many of the factors present in transplant recipients: therapy with anti-lymphocyte antibodies in cytotoxic drugs, allogeneic reactions, a systemic infection and inflammation. Thus, systemic inflammation accompanied by the release of tumor necrosis factor and other inflammatory cytokines stimulates a variety of intracellular messengers (e.g. the nuclear transcription factor NF-κB), which may initiate reactivation of HCMV from latency and resulting viral replication. Replication of HCMV is highly cell-associated, with the key host defense being MHC-linked, virus specific cytotoxic T lymphocytes. Different forms of immuno-suppression used in organ transplantation affect different aspects of viral infection; anti-lymphocyte antibodies and cytotoxic drugs enhance viral activation from latency, whereas cyclosporen, tacolimus, and corticosteroids promote the persistence and spread of virus by direct effects on viral replication and by suppressing the host's antiviral immune responses. The myriad indirect effects of HCMV in transplant recipients are explained by the following observations: the virus replicates in a wide variety of cell types including epithelial cells, endothelial cells, hepatocytes, lymphocytes especially mononuclear cells and a variety of parenchymal cells. HCMV activates cellular DNA, mRNA and protein synthesis, resulting in the production of Fc receptors, intercellular adhesion molecules (vasular-cell adhesion molecules and intercellular adhesion molecules), cellular oncogenes (myc and fos), a cell-surface glycoprotein homologous II MHC class I antigens, and a variety of pro-inflammatory cytokines. These cytokines enhance the display of endothelial cell MHC class II antigens in the allograft. In addition, HCMV blocks the processing and display of HCMV specific early antigens, protecting HCMV-infected cells from cytotoxic cellular immune response. Moreover, HCMV induces production of different cytokines and chemokines causing modulation of the immune system. As a result of HCMV-mediated immune deficits, the patient is rendered more susceptible to opportunistic infections. The prevention of HCMV infection is of great importance. Although there is no consensus about the regimen of prophylaxis against HCMV three points are worth emphasizing: Firstly, the intensity of prophylaxis must be proportional to the intensity of immuno-suppression and to the risk of viral reactivation. Secondly, prophylaxis must be prevent or limitate reactivation of the virus and block HCMV replication at immediate early stage of replication to avoid pathogenic and immune modulatory effects caused by immediate early and early gene products of the virus. Thirdly, to prevent relapse after premature prophylaxis, effective anti-viral prophylaxis with negative surveillance studies must be maintained for at least three months (The New England Journal of Medicine, J. A. Fishman et al., volume 338, No. 24 pp. 1741-1751).

Chemotherapy of HCMV infection/disease knows three admitted medicaments: Ganciclovir, Foscarnet and Cidofovir. It is the main aim of therapy to hinder the establishment of HCMV disease or to reduce the severity of an HCMV infection.

Ganciclovir (GCV): Ganciclovir is a deoxyguanosine-analogue which is phosphorylated by UL97 a phosphor transferase of the virus. It is thus activated. Cellular kinases phosphorylate GCV monophosphate to di- and tri-phosphate which may serve as a competitive inhibitor of the HCMV polymerase during DNA replication, thus leads to the abortion of the DNA chain elongation. Ganciclovir is introduced either intravenously or orally.

Foscarnet (Foscavir, FCV): Foscarnet is a pyrophosphate analogue which inhibits the HCMV-polymerase by blocking the pyrophosphate binding site and thereby inhibiting the breakdown of deoxynucleosidetriphosphate to deoxynucleosidemonophosphate and pyrophosphate. The inhibition is reversible and non-competitive. In contrast to GCV, Foscarnet must not be activated and it is not incorporated into the viral DNA chain.

Cidofovir (CDV): Cidofovir is a non-cyclic nucleotide phosphonate. Cellular enzymes phosphorylate this substance to create di-phosphorylate derivative which acts as an inhibitor of the HCMV DNA-polymerase. Cidofovir-phosphonate is also incorporated into the DNA chain. This leads to termination in DNA replication.

Transplant recipients presently receive primarily GCV, whereas Foscarnet is rarely used due to its toxicity (e.g. nephrotoxin). Cidofovir and Foscarnet are being used for HCMV retinitis in AIDS patients. In particular for those patients which have developed a GCV resistance. Apart from the anti herpetics mentioned above, anti-HCMV-hyperimmunoglobulins and highly dosed Acyclovir is being administered to patients for prophylactic treatment. Such prophylactic treatment is being discussed controversially. In a meter analysis by Bass et al. (Bass, E B, Powe. N R, Godman, S N, Graziano, S L, Griffith, R I, Kickler, T S, Wingard, J R (1993) Efficacy of immune globuline in preventing complications of bone marrow transplantation: A meta-analysis. Bone Marrow Transplant 12:273-282) it became apparent that HCMV reactivation and infection in bone marrow recipients cannot be stopped by application of hyperimmunoglobulines.

One can conclude that blocking the viral DNA replication and late gene expression with agents such as Ganciclovir and Foscarnet would leave the infected cell relatively immune to killing by both humoral and cell-mediated factor mechanisms. In fact, virus infected cells under therapy are relatively resistant to immune attacks. The natural killer cell response against HCMV-target cells may be inhibited. Since infected “blocked” cells would not undergo the virally induced lysis associated with a productive HCMV infection, they could persist, relatively unchallenged by the immune response, during periods of antiviral treatment. HCMV infected cells treated with GCV, FCV or CDV are able to express immediate early and early proteins known to be involved in viral pathogenesis, viral-induced immune modulation as well as immune escape of the virus-infected cell. To prevent these effects it would desirable to have therapeutics which are able to block viral replication at immediate early stage to inhibit synthesis of immediate early and early genes involved in the processes mentioned above.

In light of the state of the art, there is therefore a need for a medicament for the treatment of primarily human patients who are latently infected with HCMV and those receiving organs/cells from latently infected donors and under risk to develop severe HCMV associated disease. Ideally, this medicament would act on the latent HCMV infected cell, to prevent reactivation of the virus from latency and expression of the immediate early genes. Furthermore, there is a need for a medicament able to block HCMV replication at an immediate early (IE) or early stage of replication to avoid pathogenic, immune modulatory and immune escape effects of the virus. To date, there is no medicament for clinical administration able to block IE and early gene expression of the virus which occurs before initiation of viral DNA replication which is inhibited by GCV, FCV or CDV. Additionally, medicaments such as Ganciclovir, Foscarnet or Cidoforvir are known to be strong cytotoxins and thus have side effects in said patients. A very recent problem is the occurrence of drug resistant HCMV strains. Resistance of HCMV to antiviral agents is a well-documented complication of long-term anti-viral therapy. This problem has been observed mostly in patients with AIDS and HCMV retinitis, in whom drug resistant HCMV infections have been associated with clinical progression and therapeutic failure. GCV resistant HCMV strains are now also observed in long-term treated transplant recipients. (Erice A. 1999. Resistance of human cytomegalovirus to antiviral drugs. Clin. Microbiol. Rev. 12:286)

The present invention provides for medicaments for the treatment of HCMV infected patients acting primarily on infected cells thereby accomplishing the maintenance of the virus in its latent state, and thus inhibiting its reactivation following, e.g. systemic inflammation, stress and application of cAMP-elevating drugs. Additionally, the medicament should block viral replication in permissive cells like fibroblasts, type II cells, endothelial cells, hepatocytes, smooth muscle cells to prevent pathogenic and immune modulatory effects of the virus occurring independently from viral DNA replication and production of infectious virus.

### DETAILED DESCRIPTION OF THE INVENTION

Monocytes (CD14+) and its undifferentiated monocyte/granulocyte progenitor cells (CD34+, CD33+) in the bone marrow have been identified as at least one main site of HCMV latency in human (Taylor-Wiedeman et al., 1991, Mendelson et al. 1996, Kondo et al. 1996, Sindre et al., 1996 Hahn et al., 1998, Söderberg et al., 1997a). As the half-life of monocytes in the peripheral blood is restricted to 1 to 2 days, and first HCMV antigen positive monocytes are detectable not earlier than 5 to 10 days after maximum levels of plasma TNFα supports that latency occurs in progenitor cell and not in differentiated monocytes (Fietze et al., 1994, Döcke et al., 1994, Prösch et al., 1998).

Clinical studies indicated a strong correlation between TNFα plasma levels and the incidence of HCMV (re)activation (Fietze et al., 1994, Döcke et al., 1994, Mutimer et al., 1997, Kutza et al., 1998, Asadullah et al., 1999). The proposed role of TNFα for HCMV reactivation in undifferentiated monocyte/granulocyte progenitor cells was supported by the observation that TNFα can induce HCMV IE1 transcription in in vitro latently infected human monocyte/granulocyte progenitor cells (Hahn et al., 1998). Similarly, Hummel and co-workers (Koffron et al., 1999, Hummel et al., 2001) could induce IE1 transcription in lung cells of mice latently infected with MCMV (mouse cytomegalovirus) by application of TNFα in the absence of immuno-suppression (in mice the lung represents one organ of MCMV latency). Söderberg-Naucler and her co-workers (1997b and 1998) proposed that interferon gamma and TNFα are involved in reactivation of latent virus from monocytic cells by allogenic stimulation, however, they could not demonstrate a direct effect of TNFα on virus replication.

TNFα (via NF-κB) activation was shown to stimulate the HCMV IE1/2 enhancer/promoter in undifferentiated HL-60 cells serving as a model for monocyte/granulocyte progenitor cells in a concentration-dependent manner (Stein et al., 1993, Fietze et al., 1994). The effect was specific as it could be abrogated completely by pre-incubation of TNFα with a monoclonal antibody recognising TNFα. Using the same experimental conditions we could now show that proteasome inhibitors reduce TNFα stimulation of the IE1/2 enhancer/promoter in concentrations not cytotoxic for HL-60 cells. Moreover, proteasome inhibitors are able to reduce the basal, NF-κB-independent activity of the IE1/2 enhancer/promoter at concentrations not toxic for HL-60 cells.

More importantly, proteasome inhibitors significantly reduce HCMV replication in permissively infected embryonal lung fibroblasts representing one of the target cells of HCMV in vivo. Inhibition of virus replication is associated with significant decrease in E1 and IE2 and early protein expression. Synthesis of viral late proteins, representing viral structure proteins are also significantly reduced.

Proteasomes are large multimeric and multicatalytic proteinase complexes located in the nucleus and cytosol of all eukaryotic cells which are responsible for ATP-dependent as well as ATP-independent degradation of ubiquinated proteins [Coux O, Tanaka K & Goldberg A L. Structure, and functions of the 20S, and 26S proteasomes. Ann Rev Biochem 1996; 65:2165-2171.], They catalyse key events in cell cycle and transcription regulation as well as apoptosis. Peptides inhibiting the various catalytic activities of the proteasome and synthetic derivatives of the natural proteasome inhibitor lactacystin have been widely and successfully used both in vitro and in vivo for cell biological studies as well as treatment of various forms of cancer [recently rev. in Rivett A J & Gardner R C. Proteasome inhibitors: from in vitro uses to clinical trials. J Pept Sci 2000,6:478-488, Goldberg A L & Rock K. Not just research tools—proteasome inhibitors offer therapeutic promise. Nature Medicine 2002; 8:338-340.]. Recently, the therapeutic feasibility of proteasome inhibition has been demonstrated in animal models for inflammation-associated diseases like rheumatoid arthritis, asthma, multiple sclerosis, stroke, psoriasis and vascular restenosis [Goldberg A L & Rock K. Not just research tools—proteasome inhibitors offer therapeutic promise. Nature Medicine 2002; 8:338-340; Palombella V J, Conner E M, Fuseler J W, Destree A, Davis J M, Laroux F S, Wolf R E, Huang J, Brand S, Elliott P J, Lazarus D, McCornack T, Parent L, Stein R, Adams J & Grisham M B. Role of the proteasome and NF-KB in streptococcal cell wall-induced polyarthritis. Proc Natl Acad Sci USA 1998; 95:15671-15676; Elliott P J, Pien C S, McCormack T A, Chapman I D & Adams J. Proteasome inhibition: A novel mechanism to combat asthma. J Allergy Clinlmmunol 1999; 104:294-300; Vanderiugt C L, Rahbe S M, Elliott P J, dalCanto M C & Miller S D. Treatment of established relapsing experimental autoimmune encephalomyelitis with the proteasome inhibitior PS-519. J Autimmun 2000; 14:205-211; Zhang L, Zhang Z G, Zhang R L, Lu M, Adams J, Elliott P J & Chopp M. Postischemic (6-hour) treatment with recombinant human tissue plasminogen activator and proteasome inhibitor PS-519. reduces infarction in a rat model ofembolic focal cerebral ischemia. Stroke 2001; 32:2926-2931; Meiners. S, Laule M, Rother W, Guenther C, Prauka I, Muschick P, Baumann G, Kloetzel P M & Stangi K. Ubiquitin-proteasome pathway as a new target for the prevention of restenosis. Circulation 2002; 105:483-489; Zollner T M, Podda M, Pien C, Elliott P J, Kaufmann R & Boehncke W H. Proteasome inhibition reduces superantigen-mediated T cell activation and the severity of psoriasis in a SCID-hu model. J Clin Invest 2002; 109:671-679.]. The anti-inflammatory activity of proteasome inhibitors is probably related to their inhibitory activity on NF-KB activation. The eukaryotic transcription factor NF-KB plays a key role in the pathogenesis of many inflammatory and neoplastic diseases because of its central function in induction and action of many cytokines and exogenous proinflammatory stimuli. In normal unstimulated cells, the members of the NF-KB/Rel family proteins exist as hetero- or homodimeric protein complexes associated with their natural inhibitor IKB in the cytoplasm. Extracellular signals like TNFα, IL-1 or LPS activate NF-KB by destruction of IKB proteins. By activation of the IKB kinases (DCK), IKB becomes phosphorylated and subsequently polyubiquinated before degradation by the proteasome. Once free from the inhibitor protein NF-KB translocates into the nucleus where it binds to promoter regions of responsible target genes. However, the ubiquitin-proteasome pathway is not only involved in IKB degradation but is also essential for generation of the 52 kDa and 50 kDa NF-KB subunits from its inactive 100 kDa and 105 kDa precursors. Regulation of proinflammatory genes mainly occurs by the p65/p50 NF-KB heterodimer [Palombella V, Rando O, Goldberg A & Maniatis T. The ubiquitin-proteasome pathway is required for processing the NF-KB precursor protein and the activation of NF-KB. Cell 1994; 78:773-785. Silverman N & Maniatis T. NF-KB signaling pathways in mammalian and insect innate immunity. Gens & Dev 2001, 15:2321-2342. Li Q & Verma I M. NF-KB regulationin the immune system. Nature Rev immunol 2002; 2:725-734.].

The invention relates to the use of a substance or composition comprising one or more proteasome inhibitors for the manufacture of a medicament for the treatment of an individual infected with a virus selected from the group comprising varicella zoster virus, human cytomegalovirus, HHV 6 and 7, Epstein-Barr virus and HHV8.

The inventors have found out that by applying proteasome inhibitors to a progenitor-like cell line, HL-60 (ATCC No. CCL 240), it is possible to overcome TNFα-dependent stimulation of the IE1/2 enhancer/promoter. Under the same experimental conditions proteasome inhibitor 1 (PS-1) and PS-2 were tested for its influence on TNFα stimulation of the IE1/2 enhancer/promoter of HCMV AD169 and found to reduce TNFα stimulation in a concentration-dependent manner (see e.g. FIG. 2).

The formulation of therapeutic compositions and their subsequent administration is believed to be within the skill in the art. In general, for therapeutics, a patient suspected of needing such therapy is given a compound and/or inhibitor in accordance with the invention, commonly in a pharmaceutically acceptable carrier, in amounts and for periods which will vary depending upon the nature of the particular disease, its severity and the patient's overall condition. The pharmaceutical compositions may be administered in a number of ways depending upon whether local or systemic treatment is desired and upon the area to be treated. Administration may be topical (including ophthalmic, vaginal, rectal, intranasal, transdermal), oral or parenteral. Parenteral administration includes intravenous drip or infusion, subcutaneous, intraperitoneal or intramuscular injection, pulmonary administration, e.g., by inhalation or insufflation, or intrathecal or intraventricular administration. Formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable. Coated condoms, gloves and like may also be useful. Compositions for oral administration include powders or granules, suspensions or solutions in water or nonaqueous media, capsules, sachets or tablets. Thickeners, flavouring agents, diluents, emulsifiers, dispersing aids or binders may be desirable. Compositions for parenteral, intrathecal or intraventricular administration may include sterile aqueous solutions which may also contain buffers, diluents and other suitable additives.

In addition to such pharmaceutical carriers, cationic liquids may be included in the formulation to facilitate uptake is LIPOFECTIN™ (BRL, Bethesda Md.). Dosing is dependent on severity and responsiveness of the condition to be treated, with course of treatment lasting from several days to several months or until a cure is effected or a diminution of disease state is achieved. Optimal dosing schedules can be calculated from measurements of drug accumulation in the body.

Persons of ordinary skill can easily determine optimum dosages, dosing methodologies and repetition rates. Optimum dosages may vary depending on the relative potency of individual compounds and/or inhibitors, and can generally be calculated based on IC₅₀'s or EC₅₀'s or viral infectivity levels in in vitro and in vivo animal studies. For example, given the molecular weight of a compound (derived from oligonucleotide sequence and/or chemical structure) and an effective dose such as an IC₅₀, for example (derived experimentally), a dose in mg/kg is routinely calculated.

While the substance according to the invention may be used to produce medicaments for the treatment of animals, i.e. manmmals other than humans, humans are preferred.

Of the individuals infected with a herpesviridae virus the treatment of individuals infected with Human cytomegalovirus is preferred.

As outlined in great detail above populations at greatest risk of HCMV infection and HCMV-induced diseases are those undergoing organ transplantation, a septic disease and those with malignancies receiving immuno-suppressing chemotherapy and particularly patients with AIDS. In immuno-compromised hosts most severe and profound syndromes are observed when infected with HCMV, both as primary or recurring infections. Similarly, mortality and morbidity are also increased with HCMV infections in patients of this group. Accordingly, it in a preferred embodiment of the invention the use of the substance according to the invention is a use wherein the individuals to be treated have undergone organ transplantation are receiving immuno-suppressing chemotherapy, are otherwise immuno-suppressed or have AIDS. This may include pathological involvement of adrenals (75%), lung (78%), gastrointestinal tract (30%), CNS (20%) and oculus (10%) and consequently the use of the substance according to the invention for the manufacture of a medicament for the treatment of a pathology based on HCMV in said organs detailed above.

Proteasome inhibitors act in various ways. It is preferred here that the proteasome inhibitor is selected from a group comprising substances which are able to block the enzymatic activity of the 26S proteasome complex and/or block enzymatic activity of the 20S proteasome core structure. MG-132 e.g. is a cell-permeable inhibitor of the 26S proteasome which reduces the degradation of ubiquitin-conjugated proteins in mammalian cells without affecting its ATPase or isopeptidase activities. MG-132 interferes with NF-κB activation but activates cJun N-terminal kinase (JNK1), which initiates apoptosis. Proteasome inhibitor 1 (PS-1) is a reversible inhibitor of the chymotrypsin-like activity of the multicatalytic proteinase complex 20S proteasome and has been described to prevent activation of the transcription factor NF-κB in response to TNFα. Proteasome inhibitor 2 (PS-2) also function as an inhibitor of the 20 S proteasome and NF-κB activation by blocking the decay of IκBβ and IκBβ proteins. Moreover, PS-2 does not inhibit peptidyl-glutamyl-peptide hydrolyzing activity of the 20S proteasome.

In a preferred embodiment, the proteasome inhibitor is selected from a group comprising:
a) naturally occurring proteasome inhibitors comprising: peptide derivatives which have a C-terminal expoxy keton structure, β-lacton-derivatives, aclacinomycin A, lactacystin, clastolactacystein;
b) synthetic proteasome inhibitors comprising: modified peptide aldehydes such as N-carbobenzoxy-L-leucinyl-L-leucinyl-L-leucinal (also referred to as MG132 or zLLL), or the boric acid derivative of MG232, N-carbobenzoxy-Leu-Nva-H (also referred to as MG115), N-acetyl-L-leuzinyl-L-leuzinyl-L-norleuzinal (also referred to as LLnL), N-carbobenzoxy-Ile-Glu(OBut)-Ala-Leu-H (also referred to as PSI);
c) peptides comprising: an α, β,-epoxyketone-structure, vinyl-sulfones such as, carbobenzoxy-L-leucinyl-L-leucinyl-L-leucin-vinyl-sulfon or, 4-hydroxy-5-iodo-3-nitrophenylacetyl-L-leucinyl-L-leucinyl-L-leucin-vinyl-sulfon (NLVS);
d) Glyoxal- or boric acid residues such as: pyrazyl-CONH(CHPhe)CONH(CHisobutyl)B(OH)₂ and dipeptidyl-boric-acid derivatives;
e) Pinacol-esters such as: benzyloxycarbonyl(Cbz)-Leu-leuboro-Leu-pinacol-ester.

In another preferred embodiment, the proteasome inhibitor is selected from a group comprising:
a) epoxonicin (C₂₈H₈₆N₄O₇) and/or
b) eponemycin (C₂₀H₃₆N₂O₅).

In just another preferred embodiment, the proteasome inhibitor is selected from a group comprising:
a) PS-314 as a peptidyl-boric-acid derivative which is N-pyrazinecarbonyl-L-phenylalanin-L-leuzin-boric acid (C₁₉H₂₅BN₄O₄);
b) PS-519 as a β-lacton- and a lactacystin-derivative which is 1R-[1S, 4R, 5S]-1-(1-Hydroxy-2methylpropyl)-4-propyl-6-oxa-2azabicyclo[3.2.0]heptane-3,7-dione (C₁₂H₁₉NO₄);
c) PS-273 (morpholin-CONH—(CH-naphthyl)—CONH—(CH-isobutyl)—B(OH)₂) and its enantiomere;
d) PS-293;
e) PS-296 (8-quinolyl-sulfonyl-CONH—(CH-napthyl)—CONH(—CH-isobutyl)—B(OH)₂);
f) PS-303 (NH₂(CH-naphthyl)—CONH—(CH-isobutyl)—B(OH)₂;
g) PS-321 as (morpholin-CONH—(CH-napthyl)—CONH—(CH-phenylalanin)—B(OH)₂);
h) PS-334 (CH₃—NH—(CH-naphthyl-CONH—(CH-Isobutyl)—B(OH)₂);
i) PS-325 (2-quinol-CONH—(CH-homo-phenylalanin)—CONH—(CH-isobutyl)—B(OH)₂;
j) PS-352 (phenyalanin-CH₂—CH₂—CONH—(CH-isobutyl)1—B(OH)₂;
k) PS-383 (pyridyl-CONH—(CHₚF-phenylalanin)—CONH—(CH-isobutyl)—B(OH)₂);
l) PS-341; and
m) PS-1 Z-Ile-Glu(OtBu)-Ala-Leu-CHO; PS-2 [Benzyloxycarbonyl)-Leu-Leu-phenylalaninal or Z-LLF-CHO or Z-Leu-Leu-Phe-CHO PS-1.

In another preferred embodiment, the proteasome inhibitors is selected from a group comprising:
a) PS-341 and
b) PS-1 Z-Ile-Glu(OtBu)-Ala-Leu-CHO; PS-2 [Benzyloxycarbonyl)-Leu-Leu-phenylalaninal or Z-LLF-CHO or Z-Leu-Leu-Phe-CHO PS-1.
c) PS-519 as a β-lacton- and a lactacystin-derivative which is 1R-[1S, 4R, 5S]-1-(1-Hydroxy-2methylpropyl)-4-propyl-6-oxa-2azabicyclo[3.2.0]heptane-3,7-dione (C₁₂H₁₉NO₄)

Further proteasome inhibitors according to the invention are also listed in Table 1.

The present invention also encompasses a method of treating or preventing a viral infection in a subject, the method comprising administering to the subject an amount of a compound or substance selected from the group comprising proteasome inhibitors, wherein the viral infection is an infection of varicella zoster virus, human cytomegalovirus, human herpesvirus 6 and 7, Epstein-Barr virus and Karposi's sarcoma herpesvirus. While the inventors believe the substance according to the invention may be used to treat animals, i.e. mammals other than humans, humans are preferred.

The treatment is performed with a proteasome inhibitor selected from a group comprising:
naturally occurring proteasome inhibitors comprising:
peptide derivatives which have a C-terminal expoxy keton structure, β-lacton-derivatives, aclacinomycin A, lactacystin, clastolactacystein;
synthetic proteasome inhibitors comprising:

modified peptide aldehydes such as N-carbobenzoxy-L-leucinyl-L-leucinyl-L-leucinal (also referred to as MG132 or zLLL or Z-Leu-Leu-Nva-CHO), or the boric acid derivative of MG132, N-carbobenzoxy-Leu-Nva-H (also referred to as MG115), N-acetyl-L-leuzinyl-L-leuzinyl-L-norvalinal (also referred to as Z-LL-Nva-CHO or Z-Leu-Leu-Nva-CHO), N-carbobenzoxy-Ile-Glu(OtBut)-Ala-Leu-H (also referred to as PSI);
peptides comprising:
an α, β,-epoxyketone-structure, vinyl-sulfones such as, carbobenzoxy-L-leucinyl-L-leucinyl-L-leucin-vinyl-sulfon or, 4-hydroxy-5-iodo-3-nutrophenylacetyl-L-leucinyl-L-leucinyl-L-leucin-vinyl-sulfon (NLVS);
Glyoxal- or boric acid-residues such as: pyrazyl-CONH(CHPhe)CONH(CHisobutyl)B(OH)₂ and dipeptidyl-boric-acid derivatives;
Pinacol-esters such as: benzyloxycarbonyl(Cbz)-Leu-leuboro-Leu-pinacol-ester;
epoxormicin (C₂₈H₈₆N₄O₇) and eponemycin (C₂₀H₃₆N₂O₅);
PS-314 as a peptidyl-boric-acid derivative which is N-pyrazinecarbonyl-L-phenylalanin-L-leuzin-boric acid (C₁₉H₂₅BN₄O₄);
PS-519 as aβ-lacton- and a lactacystin-derivative which is 1R-[1S, 4R, 5S]-1-(1-Hydroxy-2methylpropyl)-4-propyl-6-oxa-2azabicyclo[3.2.0]heptane-3,7-dione (C₁₂H₁₉NO₄);
PS-273 (morpholin-CONH-(CH-naphthyl)—CONH—(CH-isobutyl)—B(OH)₂) and its enantiomere
PS-293;
PS-296 (8-quinolyl-sulfonyl-CONH—(CH-napthyl)—CONH(—CH-isobutyl)—B(OH)2);
PS-303 (NH₂(CH-naphthyl)—CONH—(CH-isobutyl)—B(OH)₂;
PS-321 as (morpholin-CONH—(CH-napthyl)—CONH—(CH-phenylalanin)—B(OH)₂);
PS-334 (CH₃—NH—(CH-naphthyl-CONH—(CH-Isobutyl)—B(OH)₂);
PS-325 (2-quinol-CONH—(CH-homo-phenylalanin)—CONH—(CH-isobutyl)—B(OH)₂;
PS-352 (phenyalanin-CH₂—CH₂—CONH—(CH-isobutyl)1—B(OH)₂;
PS-383 (pyridyl-CONH—(CHₚF-phenylalanin)—CONH—(CH-isobutyl)—B(OH)₂);
PS-341 and
PS-1 Z-Ile-Glu(OtBu)-Ala-Leu-CHO.
PS-2 [Benzyloxycarbonyl)-Leu-Leu-phenylalaninal or Z-LLF-CHO or Z-Leu-Leu-Phe-CHO.

Preferred proteasome inhibitors may be selected from the group comprising: a) PS-341 and b) PS-1 Z-Ile-Glu(OtBu)-Ala-Leu-CHO and c) PS-519 as a β-lacton- and a lactacystin-derivative which is 1R-[1S, 4R, 5S]-1-(1-Hydroxy-2methylpropyl)-4-propyl-6-oxa-2azabicyclo [3.2.0]heptane-3,7-dione (C₁₂H₁₉NO₄).

The invention further relates to a method of treating or preventing a viral infection in a subject, the method comprising administering to the subject an amount of a compound or substance selected from the group comprising proteasome inhibitors, wherein the viral infection is an infection of varicella zoster virus, cytomegalovirus, human cytomegalovirus, human herpesvirus 6 and 7, Epstein-Barr virus and Karposi's sarcoma herpesvirus. Incorporated herein are all embodiments concerning the use of a substance or composition comprising one or more proteasome inhibitors for the manufacture of a medicament as outlined in detail above.

### EXAMPLES

### Example 1

### Influence of Proteasome Inhibitor MG-132 on TNFα Stimulation of the HCMV IE1/2 Enhancer/Promoter in HL-60 Cells

As a model for undifferentiated monocyte/granulocyte progenitor cells HL-60, cells (ATCC No. CCL 240) expressing high levels of CD34+ but low levels of typical differentiation antigens like CD11a-c and CD14 on their surface were used. Furthermore, HL-60 cells have retained the ability, to differentiate into granulocytes and monocytes depending on the stimulus. HL-60 cells were grown in RPMI 1640 medium supplemented with 10% fetal calf serum (both certified endotoxin-free, Biochrome, Germany) at 37° C. in a 5% humidified atmosphere. The cells were shown to be mycoplasma-free by the Mycoplasma Detection Kit (Boehringer Mannheim, Germany). The cells were grown up to 1×10⁶ cells per ml. HL-60 cells were transiently transfected with the plasmid pRR55 containing the native HCMV strain Ad169 IE1/2 enhancer/promoter region between nucleotides −671 and +52 relative to the transcription start site upstream of the chloramphenicol acetyl transferase (CAT) reporter gene (Fickenscher et al., 1989). Plasmid DNA was prepared using the endofree Maxi Kit (QUIAGEN, Germany or Machery & Nagel, Germany) and stored in aliquots at −20° C. Transient transfection was performed using the DEAE transfection protocol (Stein et al., 1993). Per transfection reaction 5×10⁵ cells were washed once with FCS-free medium and then resuspendend in 250 μl transfection buffer containing 1×TBS (24 mM Tris-HCl, pH 7.4, 137 mM NaCl, 5 mM KCl, 0.7 mM CaCl₂, 0.5 mM MgCl₂, 0.6 mM Na₂HPO₄), 2.5 μg plasmid DNA (1 μg/μl ) and 5 mg/ml DEAE dextran (Pharmacia-Amersham, Germany). To avoid differences in transfection efficiency, cells for parallel probes were transfected in one reaction and splitted after transfection. Transfection occurred for 1 h at 37° C. After that the cells were washed once with FCS-free RPMI 1640 medium and resuspended in medium containing 10% FCS at a concentration of 5×10⁵ cells/5 ml. MG-132 (Calbiochemie, Germany) was dissolved in ethanol (5 μg/μl) and stored in aliqotes at −20 ° C. MG-132 was added into the medium at time zero after transfection and 1 h before TNFα (5 ng/ml, human recombinant TNFα; PAN, Germany, stored in aliquots containing 10 μg/μl at −80° C.). The cells were harvested 48 h after transfection, washed once with PBS and resuspended in 70 μl CAT buffer 1 (0.25 M Tris/HCl, pH 7.8, 0.5 mM EDTA). After 10 min of incubation on ice, cell extracts were prepared by repeated frozen/thawing of the cells (routinely 5 times), 10 min incubation at 65 ° C. to inactivate isoenzymes. Cell debris was removed by centrifugation at 12000 rpm for 10 min in an Eppendorf centrifuge. Cell extracts were measured for their protein concentrations using the method of Bradford (Bradford, 1976) and Bradford reagent (Sigma, Germany). Equal quantities of protein were used in the CAT-assay as described by Gorman et al., 1982. 20 μl lysates were incubated with [¹⁴C]chloramphenicol (Hartman Analytic, Germany) and acetyl-CoA (Sigma, Germany) at 37° C. Reaction was stopped by extraction with ethylacetat (Merck, Germany). The acetylated products were quantified after separation by thin-layer chromatography using a thin layer scintillator. MG-132 in concentration of 0.5 μg/ml abrogated the TNFα dependent stimulation of the IE1/2 enhancer/promoter nearly completely. In the presence of 0.1 μg/ml MG-132 reduced TNFα stimulation by 50% (FIG. 1). At these concentrations no cytotoxic effects on HL-60 cells were observed. MG-132 in concentrations above 0.5 μg/ml also inhibited basic, TNFα-independent activity of the IE1/2 enhancer/promoter in HL-60 cells (FIG. 10).

### MG132 Inhibits NF-kB Binding Activity in TNFa-Treated HL-60 Cells

To evaluate the effect of MG132 on NF-KB activation in TNFa-treated HL-60 cells, nuclear extracts were prepared from untreated as well as TNFa and/or MG132-treated cells and tested for its binding activity to the NF-KB consensus oligonucleotide by EMSA. The presence of 0.8 μM MG132 decreased TNFa-mediated stimulation by about 80% (FIG. 11), and NF-KB binding activity was reduced by about twofold as shown in two independent experiments (data not shown).

### Example 2

### Effect of Proteasome Inhibitor PS-1 on TNFα Stimulation of the HCMV IE1/2 Enhancer/Promoter in HL-60 Cells

Under the same experimental conditions proteasome inhibitor 1 (PS-1, Calbiochemie, Germany) and PS-2 were tested for its influence on TNFα stimulation of the IE/1/2 enhancer/promoter of HCMV AD169 and found to reduce TNFα stimulation in a concentration-dependent manner. The results for PS-1 are summarised in FIG. 2.

### Examples 3-6

### Effect of Proteasome Inhibitors MG-132 and PS-1 on HCMV Replication in Human Embryonal Lung Fibroblasts (Fi 301)

Human embryonal lung fibroblasts (HELF), one of the target cells of HCMV in vivo, are fully permissive for HCMV replication in vitro. For infection experiments the laboratory adapted strain AD169 was propagated on HELFs. Virus stocks prepared from the overlay of infected cells showing 100% cytopathic effect (CPE) by ultra centrifugation were stored in liquid N₂. Confluent HELF monolayer in 50 cm² flasks were infected with AD169 at a multiplicity of infection (M.O.I.) of 0.01. Adsorption of the virus was allowed for 1 hour at 37° C. After that the monolayer was overlayed with MEM containing 4.5% FCS. Medium was free of or substituted with the proteasome inhibitor at the indicated concentration. Virus cultures were cultivated for 5 days without changing the medium. On day five p.i. virus replication, was quantified by the number of CPE visible in inverse light microscope. For quantification cells were stained with an HCMV-specific antibody recognising the IE proteins IE1 and IE2 (clone E13, Harlan Sera-tab, Loughborough, GB, 1:5000), goat anti-mouse HRP-conjugated IgG (1:4000; Boehringer Mannheim, Indianapolis, Ind., USA) as secondary antibody and the AEC staining kit (Sigma, Germany) which gives a reed nuclear staining of infected cells.

As shown in FIG. 3 MG-132, a concentration of 0.2 μg/ml, which is non-toxic for HELFs reduces the number of CPE by two orders of magnitudes. PS-1 under the same conditions dropped virus replication by 1 to 2 order of magnitudes (not shown). Inhibition of virus replication is documented by a significantly reduced number of virus-infected cells per focus (50 vs. 7 infected cells) indicating that MG132 blocks virus spread (FIGS. 4 and 5)

### Example 7

### Proteasome Inhibitor MG-132 had no Effect on Herpes Simplex Type 1 (HSV1, HHV1) and Herpes Simplex Type 2 (HSV2, HHV2) Replication in Human Embryonal Lung Fibroblasts

Confluente HELF monolayers in 24 well tissue culture plates were infected with HSV1 strain K or HSV2 at a M.O.I. of 0.001. Virus adsorption was allowed for 1 h at 37° C. After removing the viral inoculate, cells were overlayed with MEM/E, 1% Methocel and incubated for two days at 37° C. in a 5% CO₂ atmosphere. MG-132 at the indicated concentrations was added into the medium after adsorption. CPEs were quantified by light microscopy. MG132 had no effect on HSV1 (FIG. 6) and HSV2 (not shown) replication in HELF.

### Example 8

### MG-132 Partially Inhibits IE1/2 Gene Expression as Well as Early and Late Gene Expression in HCMV AD169-Infected HELF

HELF were infected with AD169 at a M.O.I. of 1 and incubated as descibed in example 3-6. MG-132 at a concentration of 0.2 μg/ml was added after adsorption of the virus.

Cells were harvested at indicated day, washed 2 times with PBS, solubilized in 200 μl lysis buffer containing 20 mM Tris-HCL, pH 7.5, 150 mM NaCl, 1% NP40, 0.02% NaN₃, aprotinin (1 μg/ml), antipain (1 μg/ml), leupeptin (2 μg/ml), phenyl-methyl-sulfonyl fluoride (PMSF) (2 mM) (all inhibitors from Roche, Mannheim, Germany) and 2.5 mM EDTA, shaken for 1 h at 350 rpm and centrifuged for 50 min at 18,000 g and 4° C. to remove lipids and cell debris. The protein-containing supernatant was stored at −70 ° C. Protein quantification was performed using. Advanced Protein Assay Reagent (TEBU, Frankfurt, Germany). Per lane, 80 μg protein denatured for 5 min. at 95° C. were electrophoresed on a 7.5% sodium dodecyl sulfate (SDS)-polyacrylamid gel and transferred (Mini Tank Elektroblotter, OWLScientific, USA) to a cellulosenitrate membrane (Protran B A. 85, Schleicher & Schuell, Dassel, Germany). The membrane was blocked overnight in TBST (10 mM Tris HCl, pH. 7.4, 150 mM NaCl, 0.05% Tween 20) supplemented with 3% FCS. Membranes were incubated with either, mouse anti-HCMV IE antigen (recognising IE1 and IE2 protein), clone E13 (1:500; Harlan Sera-Lab, Loughborough, GB), mouse monoclonal anti-HCMV p68 late protein (1:5,000; Advanced Biotechnology, Columbia, Md., USA) or goat anti-actin antibody (1:1,000; SantaCruz Biotechnology Inc., Heidelberg, Germany). After five times washing with TBST, the blots were incubated with the second, anti-species specific antibody; goat anti-mouse HRP-conjugated IgG (1:4000; Boehringer Mannheim, Indianapolis, Ind., USA) or donkey anti-goat HPR-conjugated IgG (1:4000; SantaCruz Biotechnology, Heidelberg, Germany) for 1 h. The immunoreactive bands were visualized and quantified using SuperSignal substrate (Pierce, Rockford, Ill., USA) and a CCD-Camera (Raytest, Germany).

Blots shown in FIG. 8 demonstrate that E protein expression in MG-132 cells is reduced compared with the untreated virus control. Remarkably, the effect is stronger with respect to IE2 than to IE1 protein. Late protein expression (p68) is completely blocked in MG-132-treated-cells until day 4 p.i. while actin levels remained unchanged.

### Influence of MG132 on Viral DNA Synthesis

Studying the influence of MG132 on HCMV DNA synthesis HELF were infected with AD169 at a m.o.i of 0.01 and harvested for DNA preparation at days 1, 3, and 5 p.L The inhibitor was added at day 0 after adsorption and again at day 3 p.L Quantification of viral DNA was performed by a quantitative HCMV DNA real-time PCR determining the number of viral DNA genomes per |ˆg cellular reference DNA (histone) which was quantified in a separate parallel reaction. As demonstrated in FIG. 12 viral DNA synthesis was inhibited by MG132 in a concentration-dependent manner. In the presence of 0.8 μM MG132 the number of viral genomes was decreased by two orders of magnitudes and only slightly increased over time indicating that viral DNA synthesis was nearly completely blocked.

### MG132 Causes Cytosolic Accumulation of IKB and Reduced NF-KB Activation in Uninfected and AD169-Infected HELF

To get more inside into the mechanism of MG132-induced block of HCMV replication and to prove the role of NF-KB we investigated both IKB levels in cytosolic extracts and NF-KB binding activity in nuclear extracts from uninfected and AD169-infected HELF grown in the absence or presence of 0.8 μM MG132 for two days. In the cytosol of both untreated and AD169-infected cells no or only low levels of IKB could be detected by Western blot analysis (FIG. 13A, lanes 1 and 3). However, in MG132-treated uninfected as well as infected HELF a significant accumulation of IKB was observed (FIG. 13A, lanes 2 and 4). In nuclear extracts of untreated HELF a low constitutive NF-KB binding activity was observed by EMSA (FIG. 13B, lane 1). Addition of 0.8 uM MG132 decreased this binding activity, mainly by loss of the NF-KB p50 binding activity (FIG. 13B, lane 2). Following infection with AD169 (m.o.i.=1) an increase of NF-KB binding activity was observed (FIG. 13B, lane 3) which was again reduced in the presence of MG132 (FIG. 13B, lane 4). While NF-KB p65 binding activity in EMSA was not significantly; influenced by MG132, interestingly, a significant reduction of NF-KB p50 binding activity was observed (FIG. 13B, lanes 5 to 8). These data suggest that inhibition of NF-KB occurs by blocking of both LcB degradation and cleavage of the precursor protein p105 to generate NF-KB p50.

### MG132 Reduces HCMV-Induced ICAM-1, but not IL-6, IL-8 and RANTES Expression in HELF

Next, we wondered whether MG132 is able to reduce the immune modulatory activity of HCMV. Therefore we measured the levels of extracellular IL-6, IL-8, and RANTES in the supernatants of mock-infected and AD-169-infected HELF (m.o.i. =1) following culture in the presence or absence of increasing amounts of MG132. In parallel, surface expression of ICAM-1 (CD54) was measured by flow cytometric analysis. As expected infection of HELF with AD 169 increased secretion of IL-6, EL-8 and RANTES (data not shown) as well as surface expression of ICAM-1 over time (FIGS. 14A and B). While virus-induced secretion of IL-6, IL-8, and RANTES was not inhibited by the proteasome inhibitor, but, in some experiments even increased (data not shown), virus-induced surface expression of ICAM-1 was prevented (FIG. 14). In the presence of 0.8 uM MG132, virus-mediated up-regulation of ICAM-1 was completely abrogated while constitutive ICAM-1 expression on uninfected cells was not influenced by the drug. For comparison, GCV had no influence on virus-induced ICAM-1 expression, but rather sustained its density on the cell surface (FIG. 14, last columns).

### Cell Culture and Standard Plaque Reduction Assay

Granulocyte/monocyte progenitor like HL-60 cells (ACC CCL 240) were grown in RPMI 1640 medium supplemented with 10% foetal calf serum (FCS, both from Biochrom, Germany and certified to be endotoxin free). Permissive human embryonal lung fibroblasts (HELF) were grown as monolayer in Dulbecco's modified Eagle's medium (BioWhittaker Europa, Belgium) containing 2.0% FCS. Virus stocks of HCMV strain AD169. of laboratory-derived ganciclovir (GCV)-resistant viruses XbaF 4-3-1 and 1117′ 3-1-2 (both are kindly provided from K. K. Biron, Burroughs Wellcome Colo.), HSV 1, strain K, and HSV 2, strain DG (both are patients isolates from the strain collection of this institute) were prepared from the supernatant of infected cell cultures and stored in liquid nitrogen. For standard plaque reduction assays as well as for experiments with immunoblot analysis HELF were seeded into 25 cm² cell culture flasks. For preparation of cytosolic and nuclear extracts cells were seeded into 100 cm² culture flasks. Generally, confluent monolayers were infected at an multiplicity of infection (m.o.i.) of 0.0002 for standard plaque reduction assay and a m.o.i. of 1 for immunoblot analysis, for preparation of cytosolic and nuclear extracts as well as for measurement of cytokine, chemokine and adhesion molecules in cell culture supernatant. To study DNA synthesis HELF were seeded into 12 well dishes and infected with a m.o.i. of 0.01. For infection of the monolayers virus adsorption was allowed for 1 hour at 37° C. After adsorption the free virus was removed and cells were overlayed with fresh medium. In all experiments the proteasome inhibitors were added after virus adsorption and again at day three and five p.L Proteasome inhibitors MG132, PS I, PS II and PS III were obtained from Calbiochem, Germany. Stock solutions containing 5 mg/ml were made in ethanol for MG132 and PSI and DMSO for PS n as recommended by the manufactures and stored at −20° C. PS III stock solution of 1 mg/ml was made in methanol. To obtain working solutions stock solutions were freshly diluted in cell culture medium. Vehicle concentrations were shown to have no effect on HCMV IE1/2 enhancer/promoter activity as well as AD169 replication. Toxicity of the proteasome inhibitors were estimated by measuring cell viability and proliferation.

### Plasmids, Transfection and CAT Assay

The reporter gene plasmid pRR55 contains the whole HCMV IE1/2 enhancer/promoter region between nucleotides +52 and −637 with respect to the IE1 transcriptions start site, upstream of the chloramphenicol acetyltransferase (CAT) gene [Fickenscher H, Stamminger T, Riiger R & Fleckenstein B. The role of a repetetive palindrormic sequence element in the human cytomegalovirus major immediate early enhancer. J Gen Virol 1989; 70:107-123.]. The plasmid was kindly provided by T. Stamminger (Eriangen, Germany). For transient transfection of HL-60 cells we used the DEAE dextran method as described earlier [Stein J, Volk H D, Liebenthal C, Kruger D H & Prosch S. Tumor necrosis factor alpha stimulates the activity of the human cytomegalovirus major immediate early enhancer/promoter in immature monocytic cells. J Gen Virol 1993; 74:2333-2338]. After transfection cells were cultivated in the absence or presence of the appropriate proteasome inhibitor, which was added immediately after transfection and 1 to 2 hours before TNFa (recombinant human TNFa, 5 ng/ml, TEBU, Germany). Transfected cells were harvested 48 hours post transfection for preparation of cell extracts and CAT assay as described [Stein J, Volk H D, Liebenthal C, Kruger D H & Prosch S. Tumor necrosis factor alpha stimulates the activity of the human cytomegalovirus major immediate early enhancer/promoter in immature monocytic cells. J Gen Virol 1993; 74:2333-2338].

### Electrophoretic Mobility Shift Assay

Cytosolic and nuclear extracts from untreated and TNFa and/or MG132-treated HL-60 cells as well as uninfected and AD 169-infected HELF grown in the absence or presence of MG132 were prepared as described in detail elsewhere [Prosch S, Staak K, Stein J, Liebenthal C, Stamminger T, Volk H D & Kniger D H. Stimulation of the human cytomegalovirus IE enhancer/promoter in HL-60 cells by TNFa is mediated via induction of NF-KB. Virology 1995; 208:197-206.]. Oligonucleotides containing the consensus NF-KB binding sequence (NF-KBcons, SantaCruz Biotechnol., Germany) were radioactive labelled using “PyATP (Hartmann Anal., Germany) and oligonucleotide kinase (Roche Appl. Science, Germany). 0.1 to 1 ng radiolabeled oligonucleotide was incubated with 9 ug nuclear extract for 20 min at room temperature and separated on a 4.5% TGE polyacrylamide gel as described [Prosch S, Staak K, Stein J, Liebenthal C, Stamminger T, Volk H D & Kniger D H. Stimulation of the human cytomegalovirus IE enhancer/promoter in HL-60 cells by TNFa is mediated via induction of NF-KB. Virology 1995; 208:197-206.]. The gels were exposed to a phosphor screen. Protein binding was visualised and quantified by phosphorimaging (phosphorimager, type SI) and ImageQuant software (both from Molecular Dynamics, Amersham Pharmacia Biotech., Germany). For supershift assays the reaction, mixture was supplemented with 1.5 μl of the appropriate antibody—anti NT-XB p50 and anti NF-KB p65 (both obtained from SantaCruz Biotechnol., Germany).

### Immunoblot Analysis

For preparation of cell extracts AD169-infected (m.o.i.=1) and uninfected HELF were washed three times with phosphate-buffered saline, harvested by cell scraping, washed again and lysed with 200 μl of lysis buffer supplemented with a protease inhibitor cocktail as described [Beutler T, Hoflich C, Steven P A, Kriiger D H & Prosch S. Downregulation of the epidermal growth factor receptor by human cytomegalovirus infection in human fetal lung fibroblasts. Am J Respir Cell Mol Biol 2003; 28:86-94.]. Preparation of cytosolic and nuclear extracts occurred as described above. Protein quantification was performed using Bradford Reagent (Sigma, Germany). Aliquotes of cell lysates containing 80 ug protein or cytosolic and nuclear extracts containing 80 and 20 μg protein, respectively, were separated on a SDS-10% polyacrylamid gel and electroblotted onto nylon membrane (Schleicher & Schull, Germany). The blots were blocked by over night incubation in tTBS containing 10% bovine serum albumin. After washing twice with tTBS the blots were incubated with the appropriate antibody for 2 hours at room temperature. The antibodies used are mouse anti HCMV IE (clone 13, 1:100, Harlan Sera-Lab, GB), mouse anti HCMV major DNA binding protein pp52 (1:100, TEBU, Germany), mouse anti HCMV late p68 (1:2,000-TEBU, Germany) or rabbit anti IicB (C-21, 1:400, SantaCruz Biotechnol, Germany). Then the blots were washed 5 times with tTBS and incubated for another 2 hours with the secondary, anti-species antibody; goat anti-mouse HPR-conjugated IgG (1:4,000, Boehringer Mannheim, Ind., USA) or goat anti-rabbit HPR-conjugated IgG (1:4,000, SantaCruz Biotechnol., Germany). The immune reactive protein bands were visualised and quantified using SuperSignal substrate (Pierce, EL., USA) and a CCD-Camera (Raytest, Germany). As a loading control one of the blots of each series was stripped and probed with goat anti-actin antibody (1:1,000) and donkey anti-goat HPR-conjugated IgG (1:4,000, both from SantaCruz Biotechnol., Germany).

### Quantitative Viral DNA Analysis

AD169-infected (m.o.i.=1) and mock-infected HELF grown for 1, 3 and 5 days in the absence or presence of MG132 or GCV were washed 5 times with PBS, harvested by scraping and washed again twice with PBS. DNA was prepared using the QIAamp DNA extraction kit (Quiagen, Germany). For DNA quantification by TaqMan real-time PCR primer corresponding to the coding sequence of surface protein gpB and histone DNA were used. Quantification of viral DNA was performed by a quantitative HCMV DNA PCR determining the number of viral DNA genomes per ug cellular reference DNA (histone) which was quantified in parallel. The copy number was determined according to a laboratory standard which was tested by interlab standardisation procedures in Germany.

### IL-6 and IL-8 Measurement by Semiautomatic ELISA

For measurement of IL-6 and EL-8 cell culture supernatants were collected from mock-infected and AD169-infected HELF cultivated in the presence or absence of MG132 or GCV (m.o.i.=1) immediately after adsorption of the virus (day 0) as well as at day 1, 2, 3 and 5 p.i. and tested for IL-6 and EL-8 by Immulite™ semiautomatic. ELISA analysis as described by the manufacturer (DPC Biermann, Germany).

### RANTES ELISA

Cell culture fluids were tested for RANTES using the ELISA KIT from R&D System, Germany, following the manufacturers instructions. Measurement of cell surface expression of ICAM-1 by flow-cytometric analysis A total of 1×10⁶ AD169-infected (m.o.i.=1) and mock-infected human embryonal fibroblasts 24 and 48 h p.i. were washed twice with phosphate-buffered saline (PBS) and collected with cell dissociation solution (SIGMA, Germany), washed again with PBS and with PBS/1% FCS. The cells then were stained with monoclonal antibody CD54-FITC or FITC-conjugated isotype control immunoglobuline (Mouse IgGI) (both from Immunotech, Germany) in adequate concentrations in PBS/10% FCS for 30 min at 8° C. Incubation was followed by repeated washes with PBS/1% FCS and with PBS. Surface CD54 amounts were analysed using a FACScalibur analyser and cellquest software (Becton Dickinson, Germany). Data were calculated as A mean fluorescence intensities of CD54 and isotype control antibodies (=specific mean fluorescence intensity, MFI).

### LEGENDS TO FIGURES

FIG. 1

FIG. 1 shows results from Example 1 (means±SEM) of four independent experiments (see above). MG-123 decreased TNFα stimulation of the IE1/2 enhancer/promoter in a concentration-dependent manner between 0.5 and 0.05 μg/ml (0.81 to 0.081 μM). The IC₅₀ was determined between 0.1 and 0.2 μg/ml (0.21 to 0.42 μM).

FIG. 2

Concentrations of 0.5 to 0.1 μg/ml (0.81 to 0.16 μM) PS-1 decreases TNFα stimulation by about 60%. The IC₅₀ was determined between 0.2 and 0.3 μM in four independent experiments.

FIG. 3

In the presence of 0.2 μg/ml (0.42 μM) MG-132 virus replication was reduced by two orders of magnitudes, addition of 0.1 μg/ml (0.21 μM) MG-132 decreased virus replication by about one order of magnitude, while 0.01 μg/ml (0.016 μM) MG-132 had no effect (See also Example 3-6).

FIG. 4

MG-132 inhibits spread of the virus. In the cultures containing 0.2 μg/ml (0.42 μM) MG-123 mainly single infected cells or very small foci containing between 2 and 7 IE-positive cells were observed, indicating that virus spread in cell culture was significantly inhibited. The number of IE-positive cells per foci in cultures grown in the presence of 0.1 μg/ml (0.21 μM) MG-132 were about 50% compared with the untreated control.

FIG. 5

Representative pictures from AD 169-infected cell cultures untreated (A) or MG-132 treated (B-D) at day 5 p.i. are shown. Virus infected cells are stained using the IE1/2 specific antibody clone E13.
A: untreated virus control
B: 0.2 μg/ml MG-132
C: 0.1 μg/ml MG-132
D: 0.001 μg/ml MG-132

FIG. 6

Under the same experimental conditions as described in example 3-6 PS-1 inhibits HCMV AD169 replication also by 1 to 2 orders of magnitude. The ID₅₀ was determined between 0.16 and 0.08 μM.

FIG. 7

MG-132 at concentrations between 0.2 and 0.01 μg/ml (0.42 to 0.021 μM) had no effect on replication of HSV 1 in HELF.

FIG. 8

FIG. 8 shows Western Blot analysis of cells (HELF) infected with HCMV AD 169 and grown in the absence or presence of MG-132 (0.175 μg/ml). Blots shown demonstrate that (A) IE 1 and IE2 protein expression in MG-132 treated cells is reduced compared with the untreated virus control. Late protein expression (p68) is reduced and completely absent at day 3 p.i., while actin levels remained unchanged. (M.O.I.=1) at days 1, 3, 5 and 7 p.i. and grown in the absence or presence of MG-132 (0.175 μg/ml) (see also Experiment 8). At days 3 and 5 medium was changed and supplemented with MG-132.

FIG. 9

FIG. 9 shows the chemical structure of PS-341 and PS-519

FIG. 10

MG-132 at concentrations above 0.5 μg/ml inhibits basal activity of the HCMV IE1/2 enhancer/promoter in HL-60 cells.

FIG. 11

FIG. 11 Influence of different proteasome inhibitors on basic and TNFa-dependent activity of the HCMV IE1/2 enhancer/promoter in HL-60 cells HL60 cells were transfected with plasmid pRR55 and grown for 48 h in the absence or presence of TNFa (5 ng/ml) and increasing concentrations of the appropriate proteasome inhibitor. The proteasome inhibitor was added immediately after transfection and one hour before TNFa. The graph represents mean value (±SEM) of TNFa stimulation as measured in at least four independent experiments. TNFa stimulation was determined by the quotient of CAT expression in control cultures. (±proteasome inhibitor but without TNFa) and in TNFa-treated cultures (±proteasome inhibitor). Shown is the rate of TNFa stimulation in pRR55-transfected HL-60 cells grown in the absence or presence of PSI and 5 ng/ml TNFa.

FIG. 12

Influence of MG132 on viral DNA synthesis; HELF were infected with AD169 at a m.o.i.=1 and harvested at day 1, 3, and 5 p.i. for DNA preparation. Quantitative real time PCR for HCMV and histone DNA were performed using TaqMan. The graph shows data from one representative experiment which was repeated four times.

FIG. 13

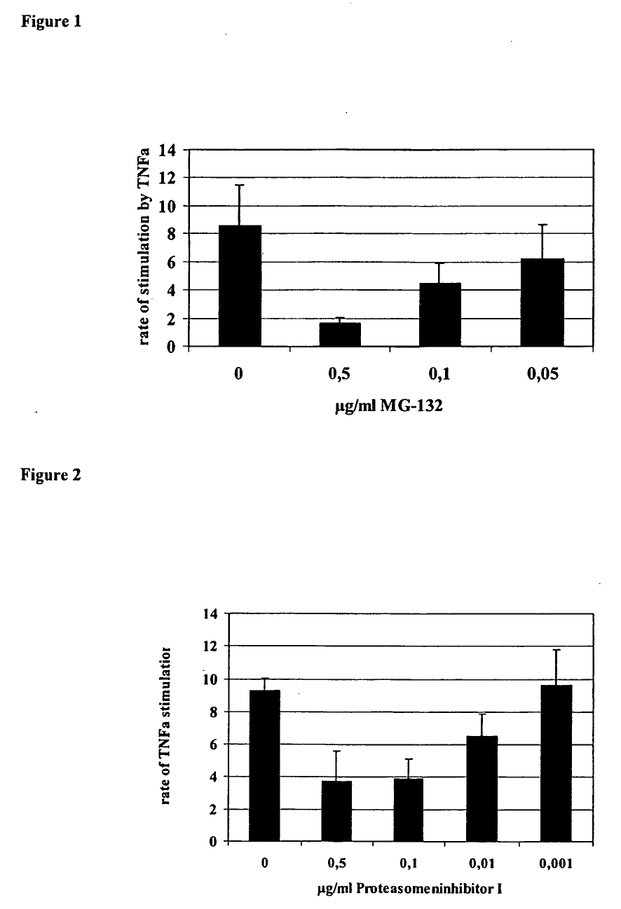

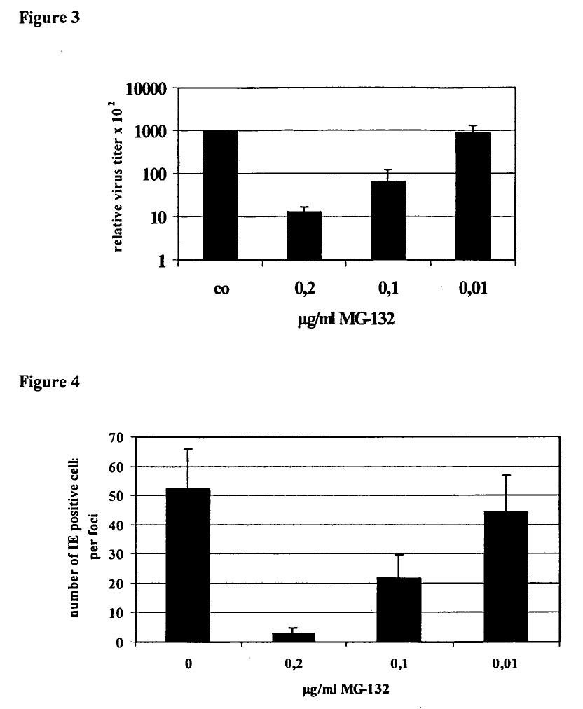

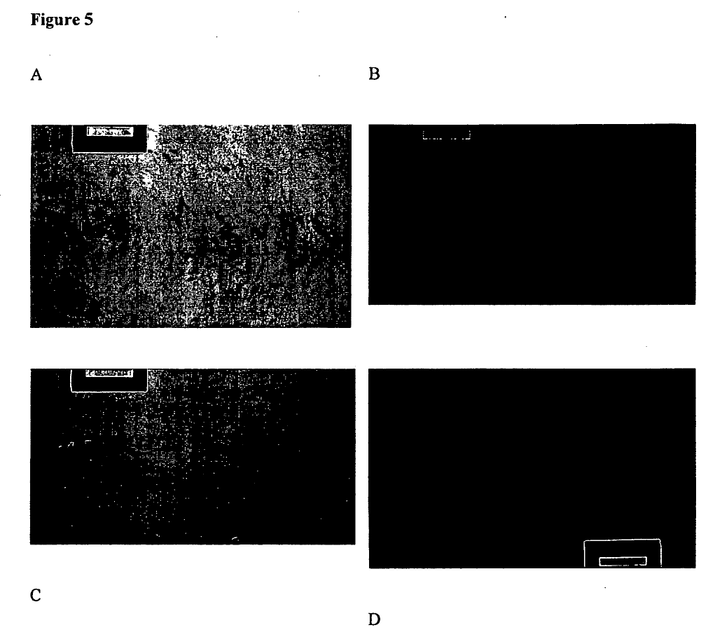

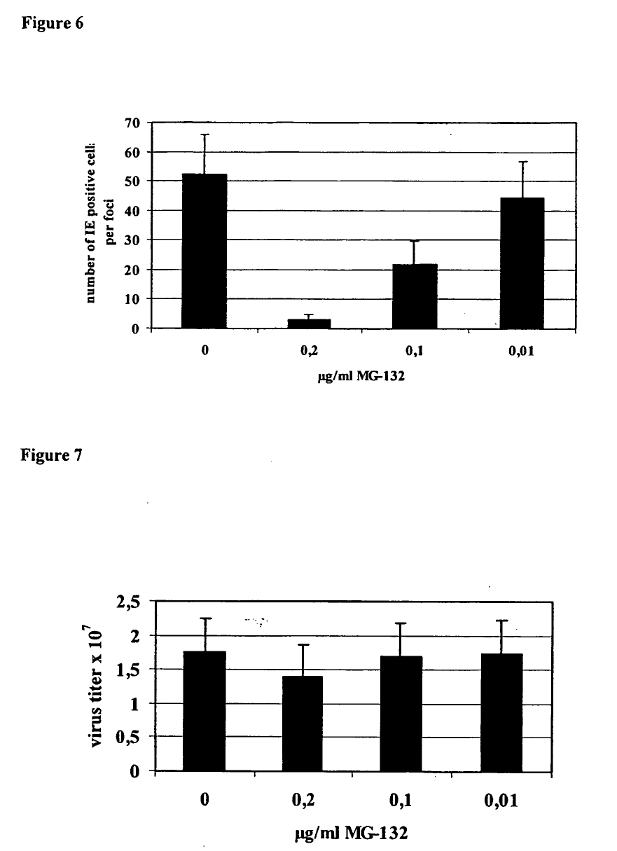

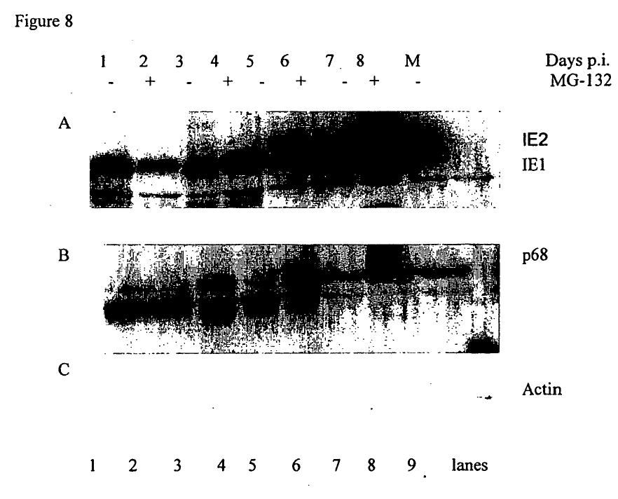

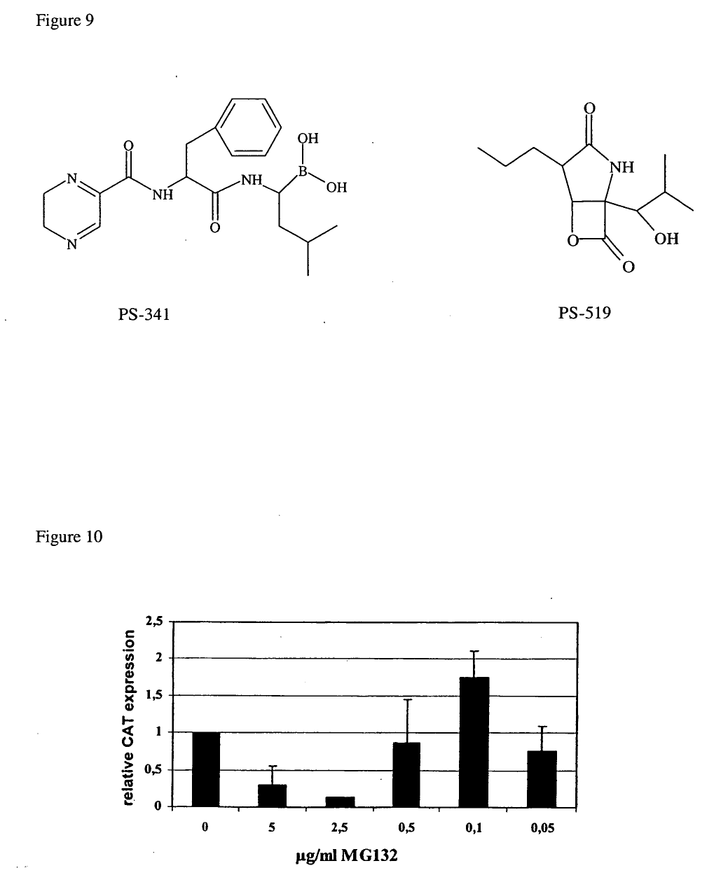

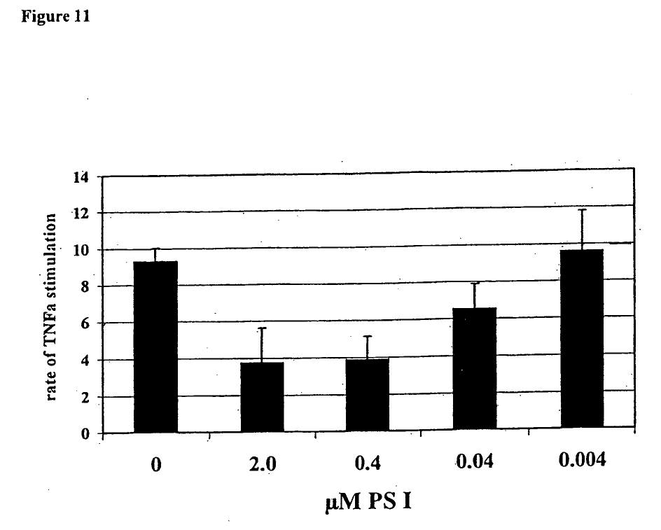

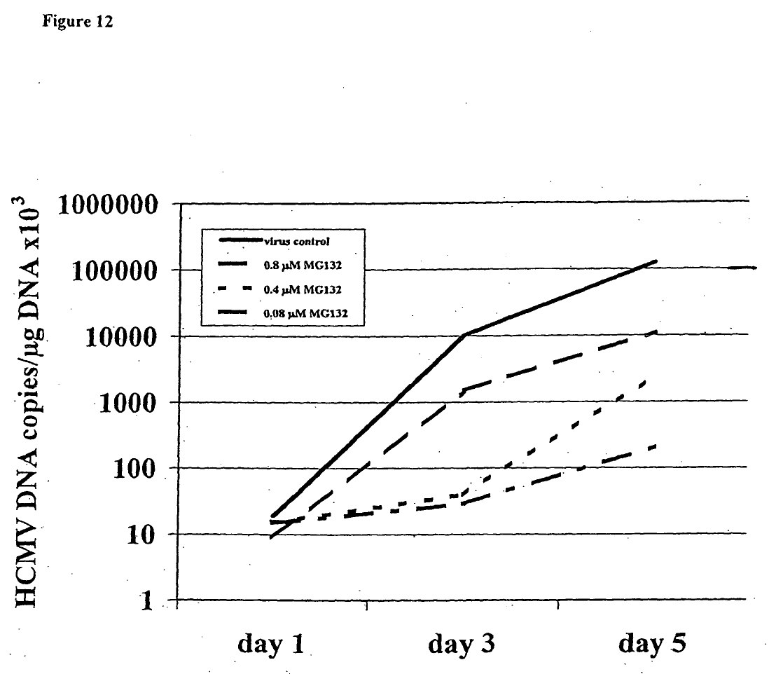

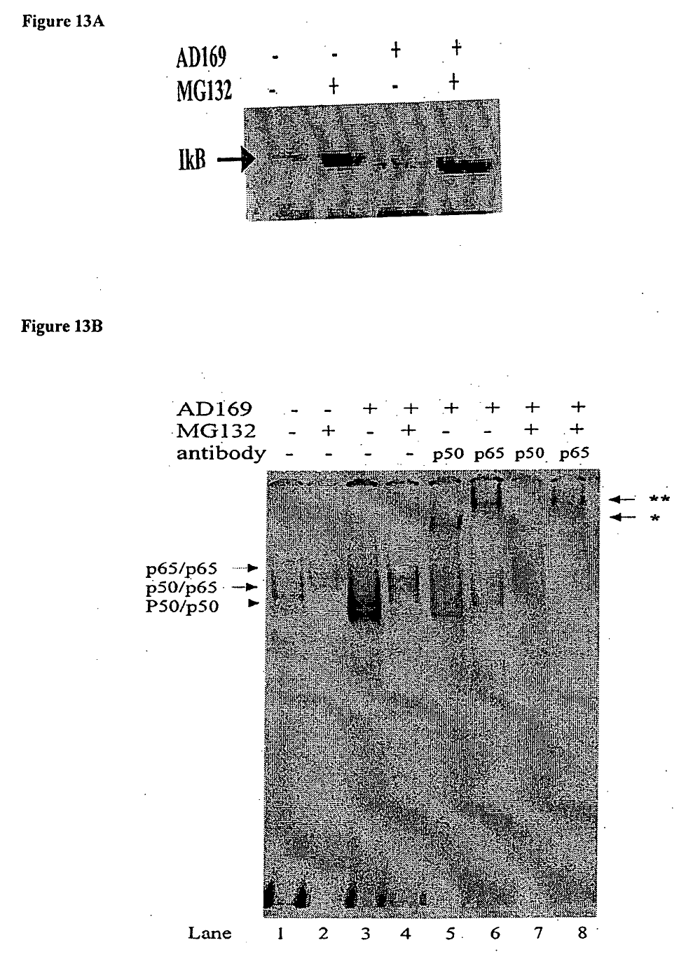

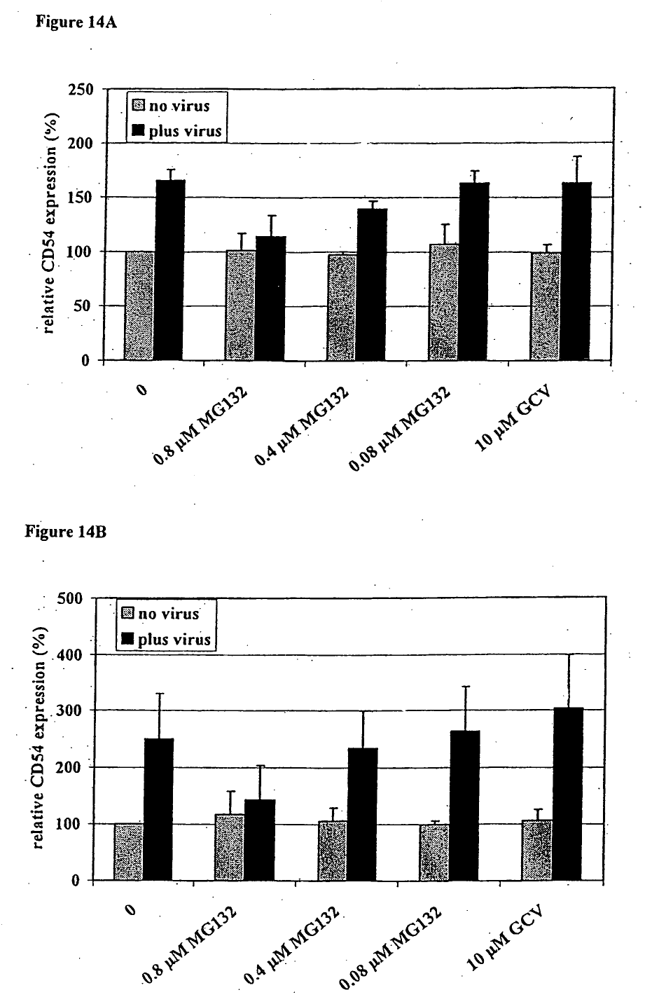

Influence of MG132 on LcBa stability (A) and NF-KB activation (B) in uninfected and AD169-infected HELF. Cytosolic and nuclear extracts were prepared from mock- and AD169-infected (moi=1) HELF 48 h p.i. grown in the absence or presence of MG 132 (0.8 μM and used for western blot analysis and EMSA, respectively.
(A) Western blot analysis of cytosolic extracts with the IKB(X specific antibody. One representative western blot which was repeated twice is demonstrated.
(B) Nuclear extracts from mock- and AD169-infected HELF grown in the absence or presence of MG132 were incubated with radiolabeled oligonucleotide NF-KBcons. The DNA-protein complexes were separated on a polyacrylamide gel; For supershift experiments the reaction mixtures were supplemented with antibodies recognizing either NF-KB p50 (lanes 6 and 8) or NF-KB p65 (lanes 5 and 7). Supershifted bands are marked by asterisks (* p50 and ** p65). Representative autoradiograph of one out of three independent experiments is shown.

FIG. 14

MG132-dependent suppression of HCMV-induced ICAM-1 surface expression Uninfected and AD169-infected HELF (m.o.i.=1) were grown in the absence or presence of MG132 or GCV and harvested 24 h (A) or 48 h (B) p.i. for surface ICAM-1 measurement by flowcytometric analysis. The specific MFI of uninfected and untreated control HELF was calculated as 100%. The graph represents mean values (±SEM) from four independent experiments.

TABLE 1
Proteasome inhibitors

| | |
|---|---|
| Molecule | References |

Proteasome inhibitors

| | |
|---|---|
| Peptide Aldehydes: | Palombella et al., 1994; Grisham et al., 1999; Jobin et al., |
| | 1998a |
| ALLnL | |
| (N-acetyl-leucinyl-leucynil-norleucynal, | |
| MG101) | |
| LLM (N-acetyl-leucinyl-leucynil-methional) | |
| Z-LLnV | Lopes et al., 1997. J. Biol. Chem. 272: 12893; Lee and |
| (carbobenzoxyl-leucinyl-leucynil- | Goldberg. 1996. J. Biol. Chem. 271: 27280; Palombella et al., |
| norvalinal, MG115) | 1994. Cell 78: 773; Rock et. al. 1994. Cell 78: 761; Vinitski et |
| | al., 1992. Biochemistry 31: 9421 |
| Z-LLL | Steinhib et al., 2001. J. Biol. Chem. 276: 4476; Merlin et al., |
| (carbobenzoxyl-leucinyl-leucynil-leucynal, | 1998. J. Biol. Chem. 273: 6373; Adams and Stein, 1996. Ann. |
| MG132) | Rev. Med. Chem. 31: 279; Klafki et al., 1996. J. Biol. Chem. |
| | 271: 28655; Lee and Goldberg, 1996; Wiertz et al., 1996. Cell |
| | 84: 769; Jensen et al., 1995. Cell 83: 129; read et al., 1995. |
| | Immunity 2: 493; Rock et al., 1994 |
| Lactacystine, b-lactone | Fenteany et al., 1998; Grisham et al., 1999 |
| Boronic Acid Peptide | Grisham et al., 1999; Iqbal et al., 1995 |
| Ubiquitin Ligase Inhibitors | Yaaron et al., 1997 |
| PS-341 | Adams, 2001 |
| Cyclosporin A | Frantz et al., 1994; Kunz et al., 1995; Marienfield et al., 1997; |
| | McCaffrey et al. 1994; Meyer et al., 1997; Wechsler et al., |
| | 1994 |
| FK506 (Tacrolimus) | Okamoto et al., 1994; Venkataraman et al., 1995 |
| Deoxyspergualin | Tepper et al., 1995 |

1. A method for treating a patient infected with a virus selected from the group consisting of varicella zoster virus, human cytomegalovirus, HHV6 and 7, Epstein-Barr virus and HHV8, comprising administering one or more proteasome inhibitors to said patient.

2. The method according to claim 1 wherein said patient is a human and said virus is human cytomegalovirus.

3. The method according to claim 1, wherein said patient has undergone organ transplantation, is receiving immuno-suppressing chemotherapy, is otherwise immuno-suppressed, has a septic disease or has AIDS.

4. The method according to claim 1, wherein said proteasome inhibitor is selected from a group consisting of substances which are able to block the enzymatic activity of the 26S proteasome complex and/or block enzymatic activity of the 20S proteasome core structure.

5. The method according to claim 1, wherein said proteasome inhibitor is selected from the group consisting of:
a) naturally occurring proteasome inhibitors,
b) synthetic proteasome inhibitors,
c) peptides,
d) Glyoxal- or boric acid residues, and
e) Pinacol-esters.

6. The method according to claim 5, wherein said naturally occurring proteasome inhibitors are selected from the group consisting of peptide derivatives which have a C-terminal expoxy keton structure, β-lacton-derivatives, aclacinomycin A, lactacystin, and clastolactacystein.

7. The method according to claim 5, wherein said synthetic proteasome inhibitors are selected from the group consisting of modified peptide aldehydes, a boric acid derivative of MG232, N-carbobenzoxy-Leu-Nva-H (also referred to as MG115), N-acetyl-L-leucinyl-L-leucinyl-L-norleucinal (also referred to as LLnL), and N-carbobenzoxy-Ile-Glu(OBut)-Ala-Leu-H (also referred to as PS-1).

8. The method according to claim 7, wherein said modified peptide aldehyde is N-carbobenzoxy-L-leucinyl-L-leucinyl-L-leucinal (also referred to as MG132 or zLLL).

9. The method according to claim 5, wherein said peptides are selected from the group consisting of an α, β,-epoxyketone-structure and vinyl-sulfones.

10. The method according to claim 9, wherein said vinyl-sulfones are selected from the group consisting of carbobenzoxy-L-leucinyl-L-leucinyl-L-leucin-vinyl-sulfon and 4-hydroxy-5-iodo-3-nitrophenylacetyl-L-leucinyl-L-leucinyl-L-leucin-vinyl-sulfon (NLVS).

11. The method according to claim 5, wherein said Glyoxal- or boric acid residues are selected from the group consisting of pyrazyl-CONH(CHPhe)CONH(CHisobutyl)B(OH)₂ and dipeptidyl-boric-acid derivatives.

12. The method according to claim 5, wherein said Pinacol-ester is benzyloxycarbonyl(Cbz)-Leu-leuboro-Leu-pinacol-ester.
